(19) United States
(12) Patent Application Publication
(10) Pub. No.: US 2025/0313892 A1
(43) Pub. Date: Oct. 9, 2025

(54) METHODS OF DECREASING BACKGROUND ON A SPATIAL ARRAY

(71) Applicant: 10x Genomics, Inc., Pleasanton, CA (US)

(72) Inventor: Marlon Stoeckius, Stockholm (SE)

(73) Assignee: 10x Genomics, Inc., Pleasanton, CA (US)

(21) Appl. No.: 19/242,371

(22) Filed: Jun. 18, 2025

Related U.S. Application Data
(63) Continuation of application No. 17/147,874, filed on Jan. 13, 2021, now Pat. No. 12,365,942.
(60) Provisional application No. 62/960,573, filed on Jan. 13, 2020.

(51) Int. Cl. C12Q 1/6874; C12Q 1/6837

(52) U.S. Cl. CPC C12Q 1/6874; C12Q 1/6837

(57) ABSTRACT

Provided herein are methods of determining a location of a target analyte in a non-permeabilized biological sample and methods of reducing background binding of an analyte on an array.

### CROSS-REFERENCE TO RELATED APPLICATIONS

This application is a continuation of U.S. patent application Ser. No. 17/147,874, filed on Jan. 13, 2021, which is claims priority under 35 U.S.C. § 119(e) to U.S. Provisional Patent Application Ser. No. 62/960,573, filed on Jan. 13, 2020, the entire contents of which are hereby incorporated by reference.

### BACKGROUND

Cells within a tissue have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling, and cross-talk with other cells in the tissue.

Spatial heterogeneity has been previously studied using techniques that typically provide data for a handful of analytes in the context of intact tissue or a portion of a tissue (e.g., tissue section), or provide significant analyte data from individual, single cells, but fails to provide information regarding the position of the single cells from the originating biological sample (e.g., tissue).

### SUMMARY

This application provides for an improvement between signal to noise (e.g., background analyte binding) during performance of any of the methods described herein for determining a location of a target analyte in a biological sample.

Provided herein are methods for decreasing background binding of a target nucleic acid on an array that include: (a) disposing a biological sample onto an array; in some embodiments, the array has a first area covered by the biological sample and a second area not covered by the biological sample; in some embodiments, the array comprises a plurality of capture probes; in some embodiments, a capture probe of the plurality of capture probes comprises a spatial barcode and a capture domain; (b) contacting the second area of the array with a solution comprising a diffusion-restricted nuclease, (c) removing the diffusion-restricted nuclease from the second area of the array; and (d) permeabilizing the biological sample, such that the capture domain binds to the target nucleic acid in the first area, thereby decreasing the background binding of a target nucleic acid on the array.

In some embodiments of any of the methods described herein, the methods further include determining (i) all or a portion of the sequence of the spatial barcode, or a complement thereof, and (ii) all or a portion of the sequence of the target nucleic acid, or a complement thereof, and using the sequences of (i) and (ii) to determine the location of the target nucleic acid in the biological sample.

In some embodiments of any of the methods described herein, the diffusion-restricted nuclease degrades single-stranded nucleic acids. In some embodiments of any of the methods described herein, the diffusion-restricted nuclease degrades double-stranded nucleic acids.

In some embodiments of any of the methods described herein, the diffusion-restricted nuclease is covalently linked to a bead, a particle, or a polymer. In some embodiments of any of the methods described herein, the polymer is a polyethylene glycol.

In some embodiments of any of the methods described herein, the diffusion-restricted nuclease is an endonuclease or an exonuclease. In some embodiments of any of the methods described herein, the exonuclease is a 3′ to 5′ exonuclease. In some embodiments of any of the methods described herein, the exonuclease is a DNAse.

In some embodiments of any of the methods described herein, the removing comprises washing.

In some embodiments of any of the methods described herein, the array comprises a slide. In some embodiments of any of the methods described herein, the array is a bead array.

In some embodiments of any of the methods described herein, the determining comprises sequencing (i) all or a portion of the sequence of the spatial barcode, or a complement thereof, and (ii) all or a portion of the sequence of the target nucleic acid, or a complement thereof. In some embodiments of any of the methods described herein, the sequencing is high throughput sequencing.

In some embodiments of any of the methods described herein, the determining comprises extending the capture probe using the target nucleic acid as the template.

In some embodiments of any of the methods described herein, steps (a) and (b) are performed at substantially the same time.

Also provided herein are methods for determining a location of a target nucleic acid in a biological sample disposed onto an array, where the array has a first area covered by the biological sample and a second area not covered by the biological sample, where the array comprises a plurality of capture probes, where a capture probe of the plurality of capture probes comprises a spatial barcode and a capture domain, wherein the methods include: (a) contacting the second area of the array with a solution comprising a diffusion-restricted nuclease; (b) removing the diffusion-restricted nuclease from the second area of the array; (c) permeabilizing the biological sample, such that the capture domain binds to the target nucleic acid; and (d) determining (i) all or a portion of the sequence of the spatial barcode, or a complement thereof, and (ii) all or a portion of the sequence of the target nucleic acid, or a complement thereof, and using the sequences of (i) and (ii) to determine the location of the target nucleic acid in the biological sample.

In some embodiments of any of the methods described herein, the diffusion-restricted nuclease degrades single-stranded nucleic acids. In some embodiments of any of the method described herein, the diffusion-restricted nuclease degrades double-stranded nucleic acids.

In some embodiments of any of the methods described herein, the diffusion-restricted nuclease is covalently linked to a bead, a particle, or a polymer. In some embodiments of the methods described herein, the polymer is a polyethylene glycol.

In some embodiments of any of the methods described herein, the diffusion-restricted nuclease is an endonuclease or an exonuclease. In some embodiments of any of the methods described herein, the exonuclease is a 3′ to 5′ exonuclease. In some embodiments of any of the methods described herein, the exonuclease is a DNAse.

In some embodiments of any of the methods described herein, the removing in step (b) comprises washing. In some embodiments of any of the methods described herein, the array comprises a slide. In some embodiments of any of the methods described herein, the array is a bead array.

In some embodiments of any of the methods described herein, the determining in step (d) comprises sequencing (i) all or a portion of the sequence of the spatial barcode, or a complement thereof, and (ii) all or a portion of the sequence of the target nucleic acid, or a complement thereof. In some embodiments of any of the methods described herein, the sequencing is high throughput sequencing.

In some embodiments of any of the methods described herein, the determining in step (d) includes extending the capture probe using the target nucleic acid as the template.

In some embodiments of any of the methods described herein, the biological sample is a tissue section. In some embodiments of any of the methods described herein, the tissue section is from a fresh frozen tissue section.

Also provided herein are methods for determining a location of a target analyte in a biological sample that include: (a) contacting a plurality of analyte capture agents to the biological sample; in some embodiments, an analyte capture agent of the plurality of analyte capture agents comprises an analyte binding moiety barcode, an analyte capture sequence, and an analyte binding moiety that binds specifically to the target analyte; (b) disposing the biological sample onto an array; in some embodiments, the array has a first area covered by the biological sample and a second area not covered by the biological sample; in some embodiments, the array comprises a plurality of capture probes; in some embodiments, a capture probe of the plurality comprises a spatial barcode and a capture domain that binds specifically to the analyte capture sequence; (c) contacting the second area of the array with a solution comprising a diffusion-restricted nuclease; (d) removing the diffusion-restricted nuclease from the second area of the array; and (e) determining (i) all or a portion of the sequence of the spatial barcode, or a complement thereof, and (ii) all or a portion of the sequence of the analyte binding moiety barcode, or a complement thereof, and using the sequences of (i) and (ii) to determine the location of the target analyte in the biological sample.

In some embodiments of any of the methods described herein, step (a) is performed before step (b). In some embodiments of any of the methods described herein, step (b) is performed before step (a).

In some embodiments of any of the methods described herein, the diffusion-restricted nuclease degrades single-stranded nucleic acids. In some embodiments of any of the methods described herein, the diffusion-restricted nuclease degrades double-stranded nucleic acids.

In some embodiments of any of the methods described herein, the diffusion-restricted nuclease is covalently linked to a bead, a particle, or a polymer. In some embodiments of any of the methods described herein, the polymer is a polyethylene glycol.

In some embodiments of any of the methods described herein, the diffusion-restricted nuclease is an endonuclease or an exonuclease. In some embodiments of any of the methods described herein, the exonuclease is a 3′ to 5′ exonuclease. In some embodiments of any of the methods described herein, the exonuclease is a DNAse.

In some embodiments of any of the methods described herein, the removing in step (d) comprises washing.

In some embodiments of any of the methods described herein, the array comprises a slide. In some embodiments of any of the methods described herein, the array is a bead array.

In some embodiments of any of the methods described herein, the determining in step (e) comprises sequencing (i) all or a portion of the sequence of the spatial barcode, or a complement thereof, and (ii) all or a portion of the sequence of the analyte binding moiety barcode, or a complement thereof. In some embodiments of any of the methods described herein, the sequencing is high throughput sequencing.

In some embodiments of any of the methods described herein, the determining in step (e) comprises extending the capture probe using the analyte binding moiety barcode as the template.

In some embodiments of any of the methods described herein, steps (a) and (b) are performed at substantially the same time.

In some embodiments of any of the methods described herein, the methods further include after step (d): permeabilizing the biological sample disposed on the array, such that the analyte binding moiety binds to the target analyte and the capture domain binds to the analyte capture sequence.

In some embodiments of any of the methods described herein, the biological sample is a tissue section. In some embodiments of any of the methods described herein, the tissue section is a fresh, frozen tissue section.

Also provided herein are kits that include an array comprising a plurality of capture probes, where a capture probe of the plurality of capture probes comprises a spatial barcode and a capture domain; and a diffusion-restricted nuclease.

Also provided herein are kits that include a plurality of analyte capture agents, where an analyte capture agent of the plurality of analyte capture agents comprises an analyte binding moiety barcode, an analyte capture sequence, and an analyte binding moiety; an array comprising a plurality of capture probes, where a capture probe of the plurality of capture probes comprises a spatial barcode and a capture domain; and a diffusion-restricted nuclease.

As used herein, the term “non-permeabilized biological sample” means a biological sample that has not been exposed to one or more permeabilizing agents (e.g., any of the exemplary permeabilizing agents described herein), or has not been subjected to a permeabilization method (e.g., any of the exemplary permeabilization methods described herein).

As used herein, the term “diffusion-restricted nuclease” means a nuclease that has is covalently and/or non-covalently attached to an agent that results in a decrease in the rate of diffusion of the nuclease as compared to the rate of diffusion of the same nuclease not covalently or non-covalently attached to the agent. In some embodiments, a diffusion-restricted nuclease is not able to significantly pass through an intact plasma membrane of a non-permeabilized mammalian cell.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The patent or application file contains at least one drawing executed in color. Copies of this patent or patent application publication with color drawing(s) will be provided by the Office upon request and payment of the necessary fee.

The following drawings illustrate certain embodiments of the features and advantages of this disclosure. These embodiments are not intended to limit the scope of the appended claims in any manner. Like reference symbols in the drawings indicate like elements.

FIG. 1 shows an example of background signal resulting from RNA in an area of an array where a brain tissue sample had not been placed.

FIG. 2 shows an example of background signal resulting from protein in an area of an array where a spleen tissue sample had not been placed.

### DETAILED DESCRIPTION

The methods described herein provide for an improvement between signal to background during performance of any of the methods described herein for determining a location of a target analyte in a biological sample.

Provided herein are methods for determining a location of a target nucleic acid in a biological sample disposed onto an array, where the array has a first area covered by the biological sample and a second area not covered by the biological sample, where the array comprises a plurality of capture probes, where a capture probe of the plurality of capture probes comprises a spatial barcode and a capture domain, where the methods include: (a) contacting the second area of the array with a solution comprising a diffusion-restricted nuclease; (b) removing the diffusion-restricted nuclease from the second area of the array; (c) permeabilizing the biological sample, such that the capture domain binds to the target nucleic acid; and (d) determining (i) all or a portion of the sequence of the spatial barcode, or a complement thereof, and (ii) all or a portion of the sequence of the target nucleic acid, or a complement thereof, and using the sequences of (i) and (ii) to determine the location of the target nucleic acid in the biological sample.

Provided herein are methods for determining a location of a target analyte in a biological sample that include: (a) contacting a plurality of analyte capture agents to the biological sample, where an analyte capture agent of the plurality of analyte capture agents comprises an analyte binding moiety barcode, an analyte capture sequence, and an analyte binding moiety that binds specifically to the target analyte; (b) disposing the biological sample onto an array, where the array has a first area covered by the biological sample and a second area not covered by the biological sample, where the array comprises a plurality of capture probes, where a capture probe of the plurality comprises a spatial barcode and a capture domain that binds to the analyte capture sequence; (c) contacting the second area of the array with a solution comprising a diffusion-restricted nuclease; (d) removing the diffusion-restricted nuclease from the second area of the array; and (e) determining (i) all or a portion of the sequence of the spatial barcode, or a complement thereof, and (ii) all or a portion of the sequence of the analyte binding moiety barcode, or a complement thereof, and using the sequences of (i) and (ii) to determine the location of the target analyte in the biological sample.

Provided herein are methods for decreasing background binding of a target nucleic acid on an array that include: (a) disposing a biological sample onto an array, where the array has a first area covered by the biological sample and a second area not covered by the biological sample, where the array comprises a plurality of capture probes, where a capture probe of the plurality of capture probes comprises a spatial barcode and a capture domain; (b) contacting the second area of the array with a solution comprising a diffusion-restricted nuclease, (c) removing the diffusion-restricted nuclease from the second area of the array; and (d) permeabilizing the biological sample, such that the capture domain binds to the target nucleic acid in the first area, thereby decreasing the background binding of a target nucleic acid on the array.

Provided herein are kits that include: an array comprising a plurality of capture probes, where a capture probe of the plurality of capture probes comprises a spatial barcode and a capture domain; and a diffusion-restricted nuclease.

Provided herein are kits that include: a plurality of analyte capture agents, where an analyte capture agent of the plurality of analyte capture agents comprises an analyte binding moiety barcode, an analyte capture sequence, and an analyte binding moiety; an array comprising a plurality of capture probes, where a capture probe of the plurality of capture probes comprises a spatial barcode and a capture domain; and a diffusion-restricted nuclease.

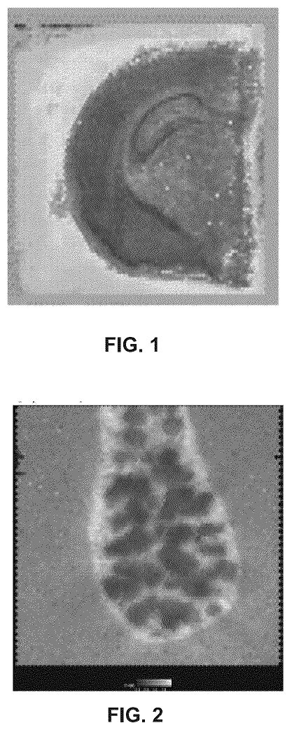

Some embodiments of any of the methods described herein can provide for at least a 0.1-fold improvement, at least a 0.5-fold improvement, at least a 0.8-fold improvement, at least a 1-fold improvement, at least a 1.5-fold improvement, at least a 2-fold improvement, at least a 2.5-fold improvement, at least a 3-fold improvement, at least a 3.5-fold improvement, at least a 4-fold improvement, at least a 4.5-fold improvement, at least a 5-fold improvement, at least a 5.5-fold improvement, at least a 6-fold improvement, at least a 6.5-fold improvement, at least a 7-fold improvement, at least a 7.5-fold improvement, at least a 8-fold improvement, at least a 8.5-fold improvement, at least a 9-fold improvement, at least a 9.5-fold improvement, at least 10-fold improvement, at least a 15-fold improvement, at least a 20-fold improvement, at least 30-fold improvement, at least 50-fold improvement, or at least 100-fold improvement in the signal to noise ratio as compared to a similar method performed without the use of a diffusion-restricted nuclease. See, e.g., FIG. 1 and FIG. 2.

Some embodiments of any of the methods described herein can provide for about a 0.1-fold improvement to about a 100-fold improvement, about a 0.1-fold improvement to about a 50-fold improvement, about a 0.1-fold improvement to about a 30-fold improvement, about a 0.1-fold improvement to about a 20-fold improvement, about a 0.1-fold improvement to about a 15-fold improvement, about a 0.1-fold improvement to about a 10-fold improvement, about a 0.1-fold improvement to about a 8-fold improvement, about a 0.1-fold improvement to about a 6-fold improvement, about a 0.1-fold improvement to about a 4-fold improvement, about a 0.1-fold improvement to about a 2-fold improvement, about a 0.1-fold improvement to about a 1-fold improvement, about a 0.1-fold improvement to about a 0.8-fold improvement, about a 0.1-fold improvement to about a 0.6-fold improvement, about a 0.1-fold improvement to about a 0.4-fold improvement, about a 0.1-fold improvement to about a 0.2-fold improvement, about a 0.2-fold improvement to about a 100-fold improvement, about a 0.2-fold improvement to about a 50-fold improvement, about a 0.2-fold improvement to about a 30-fold improvement, about a 0.2-fold improvement to about a 20-fold improvement, about a 0.2-fold improvement to about a 15-fold improvement, about a 0.2-fold improvement to about a 10-fold improvement, about a 0.2-fold improvement to about a 8-fold improvement, about a 0.2-fold improvement to about a 6-fold improvement, about a 0.2-fold improvement to about a 4-fold improvement, about a 0.2-fold improvement to about a 2-fold improvement, about a 0.2-fold improvement to about a 1-fold improvement, about a 0.2-fold improvement to about a 0.8-fold improvement, about a 0.2-fold improvement to about a 0.6-fold improvement, about a 0.2-fold improvement to about a 0.4-fold improvement, about a 0.4-fold improvement to about a 100-fold improvement, about a 0.4-fold improvement to about a 50-fold improvement, about a 0.4-fold improvement to about a 30-fold improvement, about a 0.4-fold improvement to about a 20-fold improvement, about a 0.4-fold improvement to about a 15-fold improvement, about a 0.4-fold improvement to about a 10-fold improvement, about a 0.4-fold improvement to about a 8-fold improvement, about a 0.4-fold improvement to about a 6-fold improvement, about a 0.4-fold improvement to about a 4-fold improvement, about a 0.4-fold improvement to about a 2-fold improvement, about a 0.4-fold improvement to about a 1-fold improvement, about a 0.4-fold improvement to about a 0.8-fold improvement, about a 0.4-fold improvement to about a 0.6-fold improvement, about a 0.6-fold improvement to about a 100-fold improvement, about a 0.6-fold improvement to about a 50-fold improvement, about a 0.6-fold improvement to about a 30-fold improvement, about a 0.6-fold improvement to about a 20-fold improvement, about a 0.6-fold improvement to about a 15-fold improvement, about a 0.6-fold improvement to about a 10-fold improvement, about a 0.6-fold improvement to about a 8-fold improvement, about a 0.6-fold improvement to about a 6-fold improvement, about a 0.6-fold improvement to about a 4-fold improvement, about a 0.6-fold improvement to about a 2-fold improvement, about a 0.6-fold improvement to about a 1-fold improvement, about a 0.6-fold improvement to about a 0.8-fold improvement, about a 0.8-fold improvement to about a 100-fold improvement, about a 0.8-fold improvement to about a 50-fold improvement, about a 0.8-fold improvement to about a 30-fold improvement, about a 0.8-fold improvement to about a 20-fold improvement, about a 0.8-fold improvement to about a 15-fold improvement, about a 0.8-fold improvement to about a 10-fold improvement, about a 0.8-fold improvement to about a 8-fold improvement, about a 0.8-fold improvement to about a 6-fold improvement, about a 0.8-fold improvement to about a 4-fold improvement, about a 0.8-fold improvement to about a 2-fold improvement, about a 0.8-fold improvement to about a 1-fold improvement, about a 1-fold improvement to about a 100-fold improvement, about a 1-fold improvement to about a 50-fold improvement, about a 1-fold improvement to about a 30-fold improvement, about a 1-fold improvement to about a 20-fold improvement, about a 1-fold improvement to about a 15-fold improvement, about a 1-fold improvement to about a 10-fold improvement, about a 1-fold improvement to about a 8-fold improvement, about a 1-fold improvement to about a 6-fold improvement, about a 1-fold improvement to about a 4-fold improvement, about a 1-fold improvement to about a 2-fold improvement, about a 2-fold improvement to about a 100-fold improvement, about a 2-fold improvement to about a 50-fold improvement, about a 2-fold improvement to about a 30-fold improvement, about a 2-fold improvement to about a 20-fold improvement, about a 2-fold improvement to about a 15-fold improvement, about a 2-fold improvement to about a 10-fold improvement, about a 2-fold improvement to about a 8-fold improvement, about a 2-fold improvement to about a 6-fold improvement, about a 2-fold improvement to about a 4-fold improvement, about a 4-fold improvement to about a 100-fold improvement, about a 4-fold improvement to about a 50-fold improvement, about a 4-fold improvement to about a 30-fold improvement, about a 4-fold improvement to about a 20-fold improvement, about a 4-fold improvement to about a 15-fold improvement, about a 4-fold improvement to about a 10-fold improvement, about a 4-fold improvement to about a 8-fold improvement, about a 4-fold improvement to about a 6-fold improvement, about a 6-fold improvement to about a 100-fold improvement, about a 6-fold improvement to about a 50-fold improvement, about a 6-fold improvement to about a 30-fold improvement, about a 6-fold improvement to about a 20-fold improvement, about a 6-fold improvement to about a 15-fold improvement, about a 6-fold improvement to about a 10-fold improvement, about a 6-fold improvement to about a 8-fold improvement, about a 8-fold improvement to about a 100-fold improvement, about a 8-fold improvement to about a 50-fold improvement, about a 8-fold improvement to about a 30-fold improvement, about a 8-fold improvement to about a 20-fold improvement, about a 8-fold improvement to about a 15-fold improvement, about a 8-fold improvement to about a 10-fold improvement, about a 10-fold improvement to about a 100-fold improvement, about a 10-fold improvement to about a 50-fold improvement, about a 10-fold improvement to about a 30-fold improvement, about a 10-fold improvement to about a 20-fold improvement, about a 10-fold improvement to about a 15-fold improvement, about a 15-fold improvement to about a 100-fold improvement, about a 15-fold improvement to about a 50-fold improvement, about a 15-fold improvement to about a 30-fold improvement, about a 15-fold improvement to about a 20-fold improvement, about a 20-fold improvement to about a 100-fold improvement, about a 20-fold improvement to about a 50-fold improvement, about a 20-fold improvement to about a 30-fold improvement, about a 30-fold improvement to about a 100-fold improvement, about a 30-fold improvement to about a 50-fold improvement, or about a 50-fold improvement to about 100-fold improvement, in the signal to noise ratio as compared to a similar method performed without the use of a diffusion-restricted nuclease.

Some embodiments of any of the methods described herein can provide for at least a 0.1-fold improvement, at least a 0.5-fold improvement, at least a 1-fold improvement, at least a 1.5-fold improvement, at least a 2-fold improvement, at least a 2.5-fold improvement, at least a 3-fold improvement, at least a 3.5-fold improvement, at least a 4-fold improvement, at least a 4.5-fold improvement, at least a 5-fold improvement, at least a 5.5-fold improvement, at least a 6-fold improvement, at least a 6.5-fold improvement, at least a 7-fold improvement, at least a 7.5-fold improvement, at least a 8-fold improvement, at least a 8.5-fold improvement, at least a 9-fold improvement, at least a 9.5-fold improvement, at least 10-fold improvement, at least a 15-fold improvement, at least a 20-fold improvement, at least 30-fold improvement, at least 50-fold improvement, at least 100-fold improvement in the sequencing yield per spot as compared to a similar method performed without the use of a diffusion-restricted nuclease.

Some embodiments of any of the methods described herein can provide for about a 0.1-fold improvement to about a 100-fold improvement, about a 0.1-fold improvement to about a 50-fold improvement, about a 0.1-fold improvement to about a 30-fold improvement, about a 0.1-fold improvement to about a 20-fold improvement, about a 0.1-fold improvement to about a 15-fold improvement, about a 0.1-fold improvement to about a 10-fold improvement, about a 0.1-fold improvement to about a 8-fold improvement, about a 0.1-fold improvement to about a 6-fold improvement, about a 0.1-fold improvement to about a 4-fold improvement, about a 0.1-fold improvement to about a 2-fold improvement, about a 0.1-fold improvement to about a 1-fold improvement, about a 0.1-fold improvement to about a 0.5-fold improvement, about a 0.1-fold improvement to about a 0.2-fold improvement, about a 0.2-fold improvement to about a 100-fold improvement, about a 0.2-fold improvement to about a 50-fold improvement, about a 0.2-fold improvement to about a 30-fold improvement, about a 0.2-fold improvement to about a 20-fold improvement, about a 0.2-fold improvement to about a 15-fold improvement, about a 0.2-fold improvement to about a 10-fold improvement, about a 0.2-fold improvement to about a 8-fold improvement, about a 0.2-fold improvement to about a 6-fold improvement, about a 0.2-fold improvement to about a 4-fold improvement, about a 0.2-fold improvement to about a 2-fold improvement, about a 0.2-fold improvement to about a 1-fold improvement, about a 0.2-fold improvement to about a 0.5-fold improvement, about a 0.5-fold improvement to about a 100-fold improvement, about a 0.5-fold improvement to about a 50-fold improvement, about a 0.5-fold improvement to about a 30-fold improvement, about a 0.5-fold improvement to about a 20-fold improvement, about a 0.5-fold improvement to about a 15-fold improvement, about a 0.5-fold improvement to about a 10-fold improvement, about a 0.5-fold improvement to about a 8-fold improvement, about a 0.5-fold improvement to about a 6-fold improvement, about a 0.5-fold improvement to about a 4-fold improvement, about a 0.5-fold improvement to about a 2-fold improvement, about a 0.5-fold improvement to about a 1-fold improvement, about a 1-fold improvement to about a 100-fold improvement, about a 1-fold improvement to about a 50-fold improvement, about a 1-fold improvement to about a 30-fold improvement, about a 1-fold improvement to about a 20-fold improvement, about a 1-fold improvement to about a 15-fold improvement, about a 1-fold improvement to about a 10-fold improvement, about a 1-fold improvement to about a 8-fold improvement, about a 1-fold improvement to about a 6-fold improvement, about a 1-fold improvement to about a 4-fold improvement, about a 1-fold improvement to about a 2-fold improvement, about a 2-fold improvement to about a 100-fold improvement, about a 2-fold improvement to about a 50-fold improvement, about a 2-fold improvement to about a 30-fold improvement, about a 2-fold improvement to about a 20-fold improvement, about a 2-fold improvement to about a 15-fold improvement, about a 2-fold improvement to about a 10-fold improvement, about a 2-fold improvement to about a 8-fold improvement, about a 2-fold improvement to about a 6-fold improvement, about a 2-fold improvement to about a 4-fold improvement, about a 4-fold improvement to about a 100-fold improvement, about a 4-fold improvement to about a 50-fold improvement, about a 4-fold improvement to about a 30-fold improvement, about a 4-fold improvement to about a 20-fold improvement, about a 4-fold improvement to about a 15-fold improvement, about a 4-fold improvement to about a 10-fold improvement, about a 4-fold improvement to about a 8-fold improvement, about a 4-fold improvement to about a 6-fold improvement, about a 6-fold improvement to about a 100-fold improvement, about a 6-fold improvement to about a 50-fold improvement, about a 6-fold improvement to about a 30-fold improvement, about a 6-fold improvement to about a 20-fold improvement, about a 6-fold improvement to about a 15-fold improvement, about a 6-fold improvement to about a 10-fold improvement, about a 6-fold improvement to about a 8-fold improvement, about a 8-fold improvement to about a 100-fold improvement, about a 8-fold improvement to about a 50-fold improvement, about a 8-fold improvement to about a 30-fold improvement, about a 8-fold improvement to about a 20-fold improvement, about a 8-fold improvement to about a 15-fold improvement, about a 8-fold improvement to about a 10-fold improvement, about a 10-fold improvement to about a 100-fold improvement, about a 10-fold improvement to about a 50-fold improvement, about a 10-fold improvement to about a 30-fold improvement, about a 10-fold improvement to about a 20-fold improvement, about a 10-fold improvement to about a 15-fold improvement, about a 15-fold improvement to about a 100-fold improvement, about a 15-fold improvement to about a 50-fold improvement, about a 15-fold improvement to about a 30-fold improvement, about a 15-fold improvement to about a 20-fold improvement, about a 20-fold improvement to about a 100-fold improvement, about a 20-fold improvement to about a 50-fold improvement, about a 20-fold improvement to about a 30-fold improvement, about a 30-fold improvement to about a 100-fold improvement, about a 30-fold improvement to about a 50-fold improvement, or about a 50-fold improvement to about a 100-fold improvement, in the sequencing yield per spot as compared to a similar method performed without the use of a diffusion-restricted nuclease

Additional non-limiting aspects of these methods are described herein and can be used in any combination.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434):1463-1467, 2019; Lee et al., Nat. Protoc. 10(3):442-458, 2015; Trejo et al., PLOS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10x Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain).

A “spatial barcode” is a contiguous nucleic acid segment or two or more non-contiguous nucleic acid segments that function as a label or identifier that conveys or is capable of conveying spatial information. In some embodiments, a capture probe includes a spatial barcode that possesses a spatial aspect, where the barcode is associated with a particular location within an array or a particular location on a substrate.

The “capture domain” can be an oligonucleotide, a polypeptide, a small molecule, or any combination thereof, that binds specifically to a desired analyte. In some embodiments, a capture domain can be used to capture or detect a desired analyte.

In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent.

The term “analyte binding moiety” refers to a molecule or moiety capable of binding to a macromolecular constituent (e.g., an analyte, e.g., a biological analyte). In some embodiments of any of the spatial profiling methods described herein, the analyte binding moiety of the analyte capture agent that binds to a biological analyte can include, but is not limited to, an antibody, or an epitope binding fragment thereof, a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The analyte binding moiety can bind to the macromolecular constituent (e.g., analyte) with high affinity and/or with high specificity. The analyte binding moiety can include a nucleotide sequence (e.g., an oligonucleotide), which can correspond to at least a portion or an entirety of the analyte binding moiety. The analyte binding moiety can include a polypeptide and/or an aptamer (e.g., a polypeptide and/or an aptamer that binds to a specific target molecule, e.g., an analyte). The analyte binding moiety can include an antibody or antibody fragment (e.g., an antigen-binding fragment) that binds to a specific analyte (e.g., a polypeptide).

Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21;45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein. In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two-and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### I. First and Second Areas

Some embodiments of any of the methods described herein include disposing a biological sample (e.g., a non-permeabilized biological sample) onto an array (e.g., any of the exemplary arrays described herein), where the array has a first area covered by the non-permeabilized biological sample and a second area not covered by the non-permeabilized biological sample.

The first area can represent a portion of the array that is covered by the biological sample, e.g., at least 1%, at least 2%, at least 4%, at least 6%, at least 8%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, of the total area of the array covered by the biological sample.

In some examples, the first area can represent a portion of the array that is covered by the biological sample, e.g., about 1% to about 100%, about 1% to about 95%, about 1% to about 90%, about 1% to about 85%, about 1% to about 80%, about 1% to about 75%, about 1% to about 70%, about 1% to about 65%, about 1% to about 60%, about 1% to about 55%, about 1% to about 50%, about 1% to about 45%, about 1% to about 40%, about 1% to about 35%, about 1% to about 30%, about 1% to about 35%, about 1% to about 20%, about 1% to about 15%, about 1% to about 10%, about 10% to about 100%, about 10% to about 90%, about 10% to about 80%, about 10% to about 70%, about 10% to about 60%, about 10% to about 50%, about 10% to about 40%, about 10% to about 30%, about 10% to about 20%, about 20% to about 100%, about 20% to about 90%, about 20% to about 80%, about 20% to about 70%, about 20% to about 60%, about 20% to about 50%, about 20% to about 40%, about 20% to about 30%, about 30% to about 100%, about 30% to about 90%, about 30% to about 80%, about 30% to about 70%, about 30% to about 60%, about 30% to about 50%, about 30% to about 40%, about 40% to about 100%, about 40% to about 90%, about 40% to about 80%, about 40% to about 70%, about 40% to about 60%, about 40% to about 50%, about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 70% to about 100%, about 70% to about 90%, about 70% to about 80%, about 80% to about 100%, about 80% to about 90%, or about 90% to about 100%, of the total area of the array covered by the biological sample.

The second area can represent a portion of the array that is not covered by the biological sample, e.g., at least 1%, at least 2%, at least 4%, at least 6%, at least 8%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, of the total area of the array not covered by the biological sample. In some examples, the second area can represent a portion of the array that is not covered by the biological sample, e.g., about 1% to about 100%, about 1% to about 95%, about 1% to about 90%, about 1% to about 85%, about 1% to about 80%, about 1% to about 75%, about 1% to about 70%, about 1% to about 65%, about 1% to about 60%, about 1% to about 55%, about 1% to about 50%, about 1% to about 45%, about 1% to about 40%, about 1% to about 35%, about 1% to about 30%, about 1% to about 35%, about 1% to about 20%, about 1% to about 15%, about 1% to about 10%, about 10% to about 100%, about 10% to about 90%, about 10% to about 80%, about 10% to about 70%, about 10% to about 60%, about 10% to about 50%, about 10% to about 40%, about 10% to about 30%, about 10% to about 20%, about 20% to about 100%, about 20% to about 90%, about 20% to about 80%, about 20% to about 70%, about 20% to about 60%, about 20% to about 50%, about 20% to about 40%, about 20% to about 30%, about 30% to about 100%, about 30% to about 90%, about 30% to about 80%, about 30% to about 70%, about 30% to about 60%, about 30% to about 50%, about 30% to about 40%, about 40% to about 100%, about 40% to about 90%, about 40% to about 80%, about 40% to about 70%, about 40% to about 60%, about 40% to about 50%, about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 70% to about 100%, about 70% to about 90%, about 70% to about 80%, about 80% to about 100%, about 80% to about 90%, or about 90% to about 100%, of the total area of the array not covered by the biological sample.

### II. Diffusion-Restricted Nucleases

In some embodiments, the nuclease present is a diffusion-restricted, an enzyme capable of cleaving the phosphodiester bonds between nucleotides of nucleic acids (e.g. RNA and/or DNA). In some embodiments, the nuclease degrades single-strand nucleic acids (e.g., single-stranded DNA and/or single-stranded RNA). In some embodiments, the nuclease degrades double-strand nucleic acids (e.g., double-stranded DNA, double-stranded RNA, or a double-stranded DNA/RNA molecule). In some embodiments, the nuclease is an endonuclease. In some embodiment, the nuclease is an exonuclease. In some embodiments, the nuclease is a deoxyribonuclease (DNAse). In some embodiments, the nuclease is a ribonuclease (RNAse). In some embodiments, the nuclease is a 3′ to 5′ exonuclease. In some embodiments, the nuclease is a DNAse I (e.g., bovine pancreatic DNAse I). In some embodiments, the nuclease is a DNAse II. In some embodiments, the nuclease is a wild-type DNAse. In some embodiments, the nuclease comprises an amino acid sequence that is at least 70%, 75%, 80%, 85%, 90%, 95%, 99%, or 100% identical to the amino acid sequence of a wild-type DNase. In some embodiments, the nuclease comprises at least 1, 2, 3, 4, 5, 6, 7, 8, 9, 10, 11, 12, 13, 14, 15, 16, 17, 18, 19, 20, about 30, about 40, or about 50 amino acid substitutions compared to the amino acid sequence of a wild-type DNAse. In some embodiments, the nuclease is a recombinant nuclease. In some embodiments, the nuclease is expressed in a host bacterial strain (e.g., E. coli) or a host cell (e.g., insect cell). In some embodiments, the nuclease comprises additional amino acid sequences that can facilitate recombinant nuclease expression and purification (e.g., poly-histidine tag or GST tag). In some embodiments, the recombinant nuclease is encoded by a nucleic acid sequence that is at least 70%, 75%, 80%, 85%, 90%, 95%, 99%, or 100% identical to the nucleic acid sequence encoding a wild-type DNase. In some embodiments, the recombinant nuclease is encoded by a nucleic acid sequence that has at least 1, 2, 3, 4, 5, 6, 7, 8, 9, 10, 11, 12, 13, 14, 15, 16, 17, 18, 19, 20, about 30, about 40, about 50, about 60, about 70, about 80, about 90, or about 100 mutations (e.g., point mutations) compared to the nucleic acid sequence encoding a wild-type DNAse.

In some embodiments, the diffusion-restricted nuclease is covalently conjugated or non-covalently attached to a bead. For example, a bead not having a capture probe attached but having a similar shape, dimension, material, size, coating, appearance, and/or physical properties, as compared to any of the other beads described herein.

In some embodiments, the diffusion-restricted nuclease is covalently conjugated or non-covalently attached to a particle. In some embodiments, the particle can be rigid. In some embodiments, the particle can be flexible and/or compressible. A particle can generally be of any suitable shape. Examples of particle shapes include, but are not limited to, spherical, non-spherical, oval, oblong, amorphous, circular, cylindrical, cuboidal, hexagonal, and variations thereof. Particles can be of uniform size or heterogeneous size.

In some embodiments, the particle can have a diameter or maximum dimension no larger than 100 μm (e.g., no larger than 95 μm, 90 μm, 85 μm, 80 μm, 75 μm, 70 μm, 65 μm, 60 μm, 55 μm, 50 μm, 45 μm, 40 μm, 35 μm, 30 μm, 25 μm, 20 μm, 15 μm, 14 m, 13 μm, 12 μm, 11 μm, 10 μm, 9 μm, 8 μm, 7 μm, 6 μm, 5 μm, 4 μm, 3 μm, 2 μm, or 1 μm).

In some embodiments, a plurality of particles has an average diameter no larger than 100 μm. In some embodiments, a plurality of particles has an average diameter or maximum dimension no larger than 95 μm, 90 μm, 85 μm, 80 μm, 75 μm, 70 μm, 65 μm, 60 μm, 55 μm, 50 μm, 45 μm, 40 μm, 35 μm, 30 μm, 25 μm, 20 μm, 15 μm, 14 μm, 13 μm, 12 μm, 11 μm, 10 μm, 9μm, 8 μm, 7 μm, 6 μm, 5 μm, 4 μm, 3 μm, 2 μm, or 1 μm.

In some embodiments, the volume of the particle can be at least about 1 μm³, e.g., at least 1 μm³, 2 μm³, 3 μm³, 4 μm³, 5 μm³, 6 μm³, 7 μm³, 8 μm³, 9 μm³, 10 μm³, 12 μm³, 14 μm³, 16 μm³, 18 μm³, 20 μm³, 25 μm³, 30 μm³, 35 μm³, 40 μm³, 45 μm³, 50 μm³, 55 μm³, 60 μm³, 65 μm³, 70 μm³, 75 μm³, 80 μm³, 85 μm³, 90 μm³, 95 μm³, 100 μm³, 125 μm³, 150 μm³, 175 μm³, 200 μm³, 250 μm³, 300 μm³, 350 μm³, 400 μm³, 450 μm³, μm³, 500 μm³, 550 μm³, 600 μm³, 650 μm³, 700 μm³, 750 μm³, 800 μm³, 850 μm³, 900 μm³, 950 μm³, 1000 μm³, 1200 μum³, 1400 μm³, 1600 μm³, 1800 μm³, 2000 μm³, 2200 μm³, 2400 μm³, 2600 μm³, 2800 μm³, 3000 μm³, or greater.

In some embodiments, the particle can have a volume of between about 1 μm³ and 100 μm³, such as between about 1 μm³ and 10 μm³, between about 10 μm³ and 50 μm³, or between about 50 μm³ and 100 μm³. In some embodiments, the particle can include a volume of between about 100 μm³ and 1000 μm³, such as between about 100 μm³ and 500 μm³ or between about 500 μm³ and 1000 μm³. In some embodiments, the particle can include a volume between about 1000 μm³ and 3000 μm³, such as between about 1000 μm³ and 2000 μm³ or between about 2000 μm³ and 3000 μm³. In some embodiments, the particle can include a volume between about 1 μm³ and 3000 μm³, such as between about 1 μm³ and 2000 μm³, between about 1 μm³ and 1000 μm³, between about 1 μm³ and 500 μm³, or between about 1 μm³ and 250 μm³.

The particle can include two or more cross-section(s) that can be the same or different. In some embodiments, the particle can have a first cross-section that is different from a second cross-section. The particle can have a first cross-section that is at least about 0.0001 micrometer, at least 0.001 micrometer, at least 0.01 micrometer, at least 0.1 micrometer, or at least 1 micrometer. In some embodiments, the particle can include a cross-section (e.g., a first cross-section) of at least about 1 micrometer (μm), at least about 2 μm, at least about 3 μm, at least about 4 μm, at least about 5 μm, at least about 6 μm, at least about 7 μm, at least about 8 μm, at least about 9 μm, at least about 10 μm, at least about 11 μm, at least about 12 μm, at least about 13 μm, at least about 14 μm, at least about 15 μm, at least about 16 μm, at least about 17 μm, at least about 18 μm, at least about 19 μm, at least about 20 μm, or at least about 25 μm. In some embodiments, the particle can include a cross-section (e.g., a first cross-section) of about 0.01 μm to about 25 μm, about 0.01 Tm to about 20 Tm, about 0.01 Tm to about 18 Tm, about 0.01 Tm to about 16 Tm, about 0.01 Tm to about 14 Tm, about 0.01 Tm to about 12 Tm, about 0.01 Tm to about 10 Tm, about 0.01 Tm to about 8 Tm, about 0.01 Tm to about 6 Tm, about 0.01 Tm to about 4 Tm, about 0.01 Tm to about 2 Tm, about 0.01 Tm to about 1 Tm, about 0.01 Tm to about 0.5 Tm, about 0.01 Tm to about 0.1 Tm, about 0.01 Tm to about 0.05 Tm, about 0.05 μm and 25 μm, about 0.05 Tm to about 20 Tm, about 0.05 Tm to about 18 Tm, about 0.05 Tm to about 16 Tm, about 0.05 Tm to about 14 Tm, about 0.05 Tm to about 12 Tm, about 0.05 Tm to about 10 Tm, about 0.05 Tm to about 8 Tm, about 0.05 Tm to about 6 Tm, about 0.05 Tm to about 4 Tm, about 0.05 Tm to about 2 Tm, about 0.05 Tm to about 1 Tm, about 0.05 Tm to about 0.5 Tm, about 0.05 Tm to about 0.1 Tm, about 0.1 μm and 25 μm, about 0.1 Tm to about 20 Tm, about 0.1 Tm to about 18 Tm, about 0.1 Tm to about 16 Tm, about 0.1 Tm to about 14 Tm, about 0.1 Tm to about 12 Tm, about 0.1 Tm to about 10 Tm, about 0.1 Tm to about 8 Tm, about 0.1 Tm to about 6 Tm, about 0.1 Tm to about 4 Tm, about 0.1 Tm to about 2 Tm, about 0.1 Tm to about 1 Tm, about 0.1 Tm to about 0.5 Tm, about 0.5 μm and 25 μm, about 0.5 Tm to about 20 Tm, about 0.5 Tm to about 18 Tm, about 0.5 Tm to about 16 Tm, about 0.5 Tm to about 14 Tm, about 0.5 Tm to about 12 Tm, about 0.5 Tm to about 10 Tm, about 0.5 Tm to about 8 Tm, about 0.5 Tm to about 6 Tm, about 0.5 Tm to about 4 Tm, about 0.5 Tm to about 2 Tm, about 0.5 Tm to about 1 Tm, about 1 μm and 25 μm, about 1 Tm to about 20 Tm, about 1 Tm to about 18 Tm, about 1 Tm to about 16 Tm, about 1 Tm to about 14 Tm, about 1 Tm to about 12 Tm, about 1 Tm to about 10 Tm, about 1 Tm to about 8 Tm, about 1 Tm to about 6 Tm, about 1 Tm to about 4 Tm, about 1 Tm to about 2 Tm, about 2 μm and 25 μm, about 2 Tm to about 20 Tm, about 2 Tm to about 18 Tm, about 2 Tm to about 16 Tm, about 2 Tm to about 14 Tm, about 2 Tm to about 12 Tm, about 2 Tm to about 10 Tm, about 2 Tm to about 8 Tm, about 2 Tm to about 6 Tm, about 2 Tm to about 4 Tm, about 4 μm and 25 μm, about 4 Tm to about 20 Tm, about 4 Tm to about 18 Tm, about 4 Tm to about 16 Tm, about 4 Tm to about 14 Tm, about 4 Tm to about 12 Tm, about 4 Tm to about 10 Tm, about 4 Tm to about 8 Tm, about 4 Tm to about 6 Tm, about 6 μm and 25 μm, about 6 Tm to about 20 Tm, about 6 Tm to about 18 Tm, about 6 Tm to about 16 Tm, about 6 Tm to about 14 Tm, about 6 Tm to about 12 Tm, about 6 Tm to about 10 Tm, about 6 Tm to about 8 Tm, about 8 μm and 25 μm, about 8 Tm to about 20 Tm, about 8 Tm to about 18 Tm, about 8 Tm to about 16 Tm, about 8 Tm to about 14 Tm, about 8 Tm to about 12 Tm, about 8 Tm to about 10 Tm, about 10 μm and 25 μm, about 10 Tm to about 20 Tm, about 10 Tm to about 18 Tm, about 10 Tm to about 16 Tm, about 10 Tm to about 14 Tm, about 10 Tm to about 12 Tm, about 12 μm and 25 μm, about 12 Tm to about 20 Tm, about 12 Tm to about 18 Tm, about 12 Tm to about 16 Tm, about 12 Tm to about 14 Tm, about 14 μm and 25 μm, about 14 Tm to about 20 Tm, about 14 Tm to about 18 Tm, about 14 Tm to about 16 Tm, about 16 μm and 25 μm, about 16 Tm to about 20 Tm, about 16 Tm to about 18 Tm, about 18 μm and 25 μm, about 18 Tm to about 20 Tm, or about 20 Tm to about 25 Tm.

For example, the particle can include a cross-section (e.g., a first cross-section) of about 1 μm to 100 μm. In some embodiments, the particle can have a second cross-section that is at least about 1 μm. For example, the particle can include a second cross-section of at least about 1 Tm, at least about 2 μm, at least about 3 μm, at least about 4 μm, at least about 5 μm, at least about 6 μm, at least about 7 μm, at least about 8 μm, at least about 9 μm, at least about 10 μm, at least about 11 μm, at least about 12 μm, at least about 13 μm, at least about 14 μm, at least about 15 μm, at least about 16 μm, at least about 17 μm, at least about 18 μm, at least about 19 μm, at least about 20 μm, at least about 25 μm, at least about 30 μm, at least about 35 μm, at least about 40 μm, at least about 45 μm, at least about 50 μm, at least about 55 μm, at least about 60 μm, at least about 65 μm, at least about 70 μm, at least about 75 μm, at least about 80 μm, at least about 85 μm, at least about 90 μm, at least about 100 μm, at least about 120 μm, at least about 140 μm, at least about 160 μm, at least about 180 μm, at least about 200 μm, at least about 250 μm, at least about 300 μm, at least about 350 μm, at least about 400 μm, at least about 450 μm, or at least about 500 μm. In some embodiments, the particle can include a second cross-section of about 1 μm to about 500 μm, about 1 Tm to about 450 Tm, about 1 Tm to about 400 Tm, about 1 Tm to about 350 Tm, about 1 Tm to about 300 Tm, about 1 Tm to about 250 Tm, about 1 Tm to about 200 Tm, about 1 Tm to about 150 Tm, about 1 Tm to about 100 Tm, about 1 Tm to about 80 Tm, about 1 Tm to about 60 Tm, about 1 Tm to about 40 Tm, about 1 Tm to about 20 Tm, about 1 Tm to about 15 Tm, about 1 Tm to about 10 Tm, about 1 Tm to about 8 Tm, about 1 Tm to about 6 Tm, about 1 Tm to about 4 Tm, about 1 Tm to about 2 Tm, about 2 82 m to about 500 μm, about 2 Tm to about 450 Tm, about 2 Tm to about 400 Tm, about 2 Tm to about 350 Tm, about 2 Tm to about 300 Tm, about 2 Tm to about 250 Tm, about 2 Tm to about 200 Tm, about 2 Tm to about 150 Tm, about 2 Tm to about 100 Tm, about 2 Tm to about 80 Tm, about 2 Tm to about 60 Tm, about 2 Tm to about 40 Tm, about 2 Tm to about 20 Tm, about 2 Tm to about 15 Tm, about 2 Tm to about 10 Tm, about 2 Tm to about 8 Tm, about 2 Tm to about 6 Tm, about 2 Tm to about 4 Tm, about 4 μm to about 500 μm, about 4 Tm to about 450 Tm, about 4 Tm to about 400 Tm, about 4 Tm to about 350 Tm, about 4 Tm to about 300 Tm, about 4 Tm to about 250 Tm, about 4 Tm to about 200 Tm, about 4 Tm to about 150 Tm, about 4 Tm to about 100 Tm, about 4 Tm to about 80 Tm, about 4 Tm to about 60 Tm, about 4 Tm to about 40 Tm, about 4 Tm to about 20 Tm, about 4 Tm to about 15 Tm, about 4 Tm to about 10 Tm, about 4 Tm to about 8 Tm, about 4 Tm to about 6 Tm, about 6 μm to about 500 μm, about 6 Tm to about 450 Tm, about 6 Tm to about 400 Tm, about 6 Tm to about 350 Tm, about 6 Tm to about 300 Tm, about 6 Tm to about 250 Tm, about 6 Tm to about 200 Tm, about 6 Tm to about 150 Tm, about 6 Tm to about 100 Tm, about 6 Tm to about 80 Tm, about 6 Tm to about 60 Tm, about 6 Tm to about 40 Tm, about 6 Tm to about 20 Tm, about 6 Tm to about 15 Tm, about 6 Tm to about 10 Tm, about 6 Tm to about 8 Tm, about 8 μm to about 500 μm, about 8 Tm to about 450 Tm, about 8 Tm to about 400 Tm, about 8 Tm to about 350 Tm, about 8 Tm to about 300 Tm, about 8 Tm to about 250 Tm, about 8 Tm to about 200 Tm, about 8 Tm to about 150 Tm, about 8 Tm to about 100 Tm, about 8 Tm to about 80 Tm, about 8 Tm to about 60 Tm, about 8 Tm to about 40 Tm, about 8 Tm to about 20 Tm, about 8 Tm to about 15 Tm, about 8 Tm to about 10 Tm, about 10 μm to about 500 μm, about 10 Tm to about 450 Tm, about 10 Tm to about 400 Tm, about 10 Tm to about 350 Tm, about 10 Tm to about 300 Tm, about 10 Tm to about 250 Tm, about 10 Tm to about 200 Tm, about 10 Tm to about 150 Tm, about 10 Tm to about 100 Tm, about 10 Tm to about 80 Tm, about 10 Tm to about 60 Tm, about 10 Tm to about 40 Tm, about 10 Tm to about 20 Tm, about 10 Tm to about 15 Tm, about 15 μm to about 500 μm, about 15 Tm to about 450 Tm, about 15 Tm to about 400 Tm, about 15 Tm to about 350 Tm, about 15 Tm to about 300 Tm, about 15 Tm to about 250 Tm, about 15 Tm to about 200 Tm, about 15 Tm to about 150 Tm, about 15 Tm to about 100 Tm, about 15 Tm to about 80 Tm, about 15 Tm to about 60 Tm, about 15 Tm to about 40 Tm, about 15 Tm to about 20 Tm, about 20 μm to about 500 μm, about 20 Tm to about 450 Tm, about 20 Tm to about 400 Tm, about 20 Tm to about 350 Tm, about 20 Tm to about 300 Tm, about 20 Tm to about 250 Tm, about 20 Tm to about 200 Tm, about 20 Tm to about 150 Tm, about 20 Tm to about 100 Tm, about 20 Tm to about 80 Tm, about 20 Tm to about 60 Tm, about 20 Tm to about 40 Tm, about 40 μm to about 500 μm, about 40 Tm to about 450 Tm, about 40 Tm to about 400 Tm, about 40 Tm to about 350 Tm, about 40 Tm to about 300 Tm, about 40 Tm to about 250 Tm, about 40 Tm to about 200 Tm, about 40 Tm to about 150 Tm, about 40 Tm to about 100 Tm, about 40 Tm to about 80 Tm, about 40 Tm to about 60 Tm, about 60 μm to about 500 μm, about 60 Tm to about 450 Tm, about 60 Tm to about 400 Tm, about 60 Tm to about 350 Tm, about 60 Tm to about 300 Tm, about 60 Tm to about 250 Tm, about 60 Tm to about 200 Tm, about 60 Tm to about 150 Tm, about 60 Tm to about 100 Tm, about 60 Tm to about 80 Tm, about 80 μm to about 500 μm, about 80 Tm to about 450 Tm, about 80 Tm to about 400 Tm, about 80 Tm to about 350 Tm, about 80 Tm to about 300 Tm, about 80 Tm to about 250 Tm, about 80 Tm to about 200 Tm, about 80 Tm to about 150 Tm, about 80 Tm to about 100 Tm, about 100 μm to about 500 μm, about 100 Tm to about 450 Tm, about 100 Tm to about 400 Tm, about 100 Tm to about 350 Tm, about 100 Tm to about 300 Tm, about 100 Tm to about 250 Tm, about 100 Tm to about 200 Tm, about 100 Tm to about 150 Tm, about 150 μm to about 500 μm, about 150 Tm to about 450 Tm, about 150 Tm to about 400 Tm, about 150 Tm to about 350 Tm, about 150 Tm to about 300 Tm, about 150 Tm to about 250 Tm, about 150 Tm to about 200 Tm, about 200 μm to about 500 μm, about 200 Tm to about 450 Tm, about 200 Tm to about 400 Tm, about 200 Tm to about 350 Tm, about 200 Tm to about 300 Tm, about 200 Tm to about 250 Tm, about 250 μm to about 500 μm, about 250 Tm to about 450 Tm, about 250 Tm to about 400 Tm, about 250 Tm to about 350 Tm, about 250 Tm to about 300 Tm, about 300 μm to about 500 μm, about 300 Tm to about 450 Tm, about 300 Tm to about 400 Tm, about 300 Tm to about 350 Tm, about 350 μm to about 500 μm, about 350 Tm to about 450 Tm, about 350 Tm to about 400 Tm, about 400 μm to about 500 μm, about 400 Tm to about 450 Tm, about 450 μm to about 500 μm.

In some embodiments, particles can be of a nanometer scale (e.g., particles can have a diameter or maximum cross-sectional dimension of about 10 nm to about 900 nm, about 10 nm to about 850 nm, about 10 nm to about 800 nm, about 10 nm to about 750 nm, about 10 nm to about 700 nm, about 10 nm to about 650 nm, about 10 nm to about 600 nm, about 10 nm to about 550 nm, about 10 nm to about 500 nm, about 10 nm to about 450 nm, about 10 nm to about 400 nm, about 10 nm to about 350 nm, about 10 nm to about 300 nm, about 10 nm to about 250 nm, about 10 nm to about 200 nm, about 10 nm to about 150 nm, about 10 nm to about 100 nm, about 10 nm to about 50 nm, about 50 nm to about 900 nm, about 50 nm to about 850 nm, about 50 nm to about 800 nm, about 50 nm to about 750 nm, about 50 nm to about 700 nm, about 50 nm to about 650 nm, about 50 nm to about 600 nm, about 50 nm to about 550 nm, about 50 nm to about 500 nm, about 50 nm to about 450 nm, about 50 nm to about 400 nm, about 50 nm to about 350 nm, about 50 nm to about 300 nm, about 50 nm to about 250 nm, about 50 nm to about 200 nm, about 50 nm to about 150 nm, about 50 nm to about 100 nm, about 100 nm to about 900 nm, about 100 nm to about 850 nm, about 100 nm to about 800 nm, about 100 nm to about 750 nm, about 100 nm to about 700 nm, about 100 nm to about 650 nm, about 100 nm to about 600 nm, about 100 nm to about 550 nm, about 100 nm to about 500 nm, about 100 nm to about 450 nm, about 100 nm to about 400 nm, about 100 nm to about 350 nm, about 100 nm to about 300 nm, about 100 nm to about 250 nm, about 100 nm to about 200 nm, about 100 nm to about 150 nm, about 150 nm to about 900 nm, about 150 nm to about 850 nm, about 150 nm to about 800 nm, about 150 nm to about 750 nm, about 150 nm to about 700 nm, about 150 nm to about 650 nm, about 150 nm to about 600 nm, about 150 nm to about 550 nm, about 150 nm to about 500 nm, about 150 nm to about 450 nm, about 150 nm to about 400 nm, about 150 nm to about 350 nm, about 150 nm to about 300 nm, about 150 nm to about 250 nm, about 150 nm to about 200 nm, about 200 nm to about 900 nm, about 200 nm to about 850 nm, about 200 nm to about 800 nm, about 200 nm to about 750 nm, about 200 nm to about 700 nm, about 200 nm to about 650 nm, about 200 nm to about 600 nm, about 200 nm to about 550 nm, about 200 nm to about 500 nm, about 200 nm to about 450 nm, about 200 nm to about 400 nm, about 200 nm to about 350 nm, about 200 nm to about 300 nm, about 200 nm to about 250 nm, about 250 nm to about 900 nm, about 250 nm to about 850 nm, about 250 nm to about 800 nm, about 250 nm to about 750 nm, about 250 nm to about 700 nm, about 250 nm to about 650 nm, about 250 nm to about 600 nm, about 250 nm to about 550 nm, about 250 nm to about 500 nm, about 250 nm to about 450 nm, about 250 nm to about 400 nm, about 250 nm to about 350 nm, about 250 nm to about 300 nm, about 300 nm to about 900 nm, about 300 nm to about 850 nm, about 300 nm to about 800 nm, about 300 nm to about 750 nm, about 300 nm to about 700 nm, about 300 nm to about 650 nm, about 300 nm to about 600 nm, about 300 nm to about 550 nm, about 300 nm to about 500 nm, about 300 nm to about 450 nm, about 300 nm to about 400 nm, about 300 nm to about 350 nm, about 350 nm to about 900 nm, about 350 nm to about 850 nm, about 350 nm to about 800 nm, about 350 nm to about 750 nm, about 350 nm to about 700 nm, about 350 nm to about 650 nm, about 350 nm to about 600 nm, about 350 nm to about 550 nm, about 350 nm to about 500 nm, about 350 nm to about 450 nm, about 350 nm to about 400 nm, about 400 nm to about 900 nm, about 400 nm to about 850 nm, about 400 nm to about 800 nm, about 400 nm to about 750 nm, about 400 nm to about 700 nm, about 400 nm to about 650 nm, about 400 nm to about 600 nm, about 400 nm to about 550 nm, about 400 nm to about 500 nm, about 400 nm to about 450 nm, about 450 nm to about 900 nm, about 450 nm to about 850 nm, about 450 nm to about 800 nm, about 450 nm to about 750 nm, about 450 nm to about 700 nm, about 450 nm to about 650 nm, about 450 nm to about 600 nm, about 450 nm to about 550 nm, about 450 nm to about 500 nm, about 500 nm to about 900 nm, about 500 nm to about 850 nm, about 500 nm to about 800 nm, about 500 nm to about 750 nm, about 500 nm to about 700 nm, about 500 nm to about 650 nm, about 500 nm to about 600 nm, about 500 nm to about 550 nm, about 550 nm to about 900 nm, about 550 nm to about 850 nm, about 550 nm to about 800 nm, about 550 nm to about 750 nm, about 550 nm to about 700 nm, about 550 nm to about 650 nm, about 550 nm to about 600 nm, about 600 nm to about 900 nm, about 600 nm to about 850 nm, about 600 nm to about 800 nm, about 600 nm to about 750 nm, about 600 nm to about 700 nm, about 600 nm to about 650 nm, about 650 nm to about 900 nm, about 650 nm to about 850 nm, about 650 nm to about 800 nm, about 650 nm to about 750 nm, about 650 nm to about 700 nm, about 700 nm to about 900 nm, about 700 nm to about 850 nm, about 700 nm to about 800 nm, about 700 nm to about 750 nm, about 750 nm to about 900 nm, about 750 nm to about 850 nm, about 750 nm to about 800 nm, about 800 nm to about 900 nm, about 800 nm to about 850 nm, or about 850 nm to about 900 nm.

In some embodiments, a particle has a diameter or volume that is about the diameter of a single cell (e.g., a single cell under evaluation).

In some embodiments, the diffusion-restricted nuclease is covalently conjugated to a polymer. The polymer can be a natural polymer, a synthetic polymer, or a combination of both natural and synthetic polymers. Examples of natural polymers include, without limitation, proteins, sugars such as deoxyribonucleic acid, rubber, cellulose, starch (e.g., amylose and amylopectin), polysaccharides, silks, polyhydroxyalkanoates, chitosan, dextran, collagen, carrageenan, ispaghula, acacia, agar, gelatin, shellac, sterculia gum, xanthan gum, corn sugar gum, guar gum, gum karaya, agarose, alginic acid, alginate, or natural polymers thereof. Examples of synthetic polymers include, without limitation, acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride), and/or combinations (e.g., co-polymers) thereof.

In some embodiments, the polymer can have an average molecular weight of at least about 100 Da (dalton), at least about 200 Da, at least about 300 Da, at least about 400 Da, at least about 500 Da, at least about 600 Da, at least about 700 Da, at least about 800 Da, at least about 900 Da, at least about 1 kDa, at least about 2 kDa, at least about 3 kDa, at least about 4 kDa, at least about 5 kDa, at least about 6 kDa, at least about 7 kDa, at least about 8 kDa, at least about 9 kDa, at least about 10 kDa, at least about 20 kDa, at least about 50 kDa, or at least about 100 kDa. In some embodiments, the polymer can have a molecular weight about 100 Da to about 500 Da, about 500 Da to about 1 kDa, about 1 kDa to about 5 kDa, about 5 kDa to about 20 kDa, about 20 kDa to about 100 kDa.

In some embodiments, the polymer can have a hydrodynamic radius no larger than 100 μm (e.g., no larger than 95 μm, 90 μm, 85 μm, 80 μm, 75 μm, 70 μm, 65 μm, 60 μm, 55 μm, 50 μm, 45 μm, 40 μm, 35 μm, 30 μm, 25 μm, 20 μm, 15 μm, 14 μm, 13 μm, 12 μm, 11 μm, 10 μm, 9 μm, 8 μm, 7 μm, 6 μm, 5 μm, 4 μm, 3 μm, 2 μm, or 1 μm).

In some embodiments, the diffusion-restricted nuclease is conjugated to a bead, a particle, or a polymer through non-covalent interactions, e.g., electrostatic interactions, hydrogen bonds, Van der Waals interactions, hydrophobic interactions, ion-induced dipole interactions, dipole-induced dipole interactions, or any combination thereof.

### III. Contacting the Second Area of the Array with a Solution Comprising a Diffusion-Restricted Nuclease

In some embodiments, the solution comprising a diffusion-restricted nuclease is added automatically (e.g., by a robot) or manually (e.g., by pipetting). In some embodiments, the solution comprising a diffusion-restricted nuclease is added dropwise by a pipette. In some embodiments, the solution comprising a diffusion-restricted nuclease is added to contact all or a portion of the second area of the array. In some embodiments, the solution comprising a diffusion-restricted nuclease is added to all or a portion of a surface of the non-permeabilized biological sample that is not facing or contacting the array.

In some embodiments, the solution is added vertically above the second area of the array. In some embodiments, the solution is present in liquid form, such that the second area is covered by the solution. In some alternative embodiments, the diffusion-restricted nuclease is contacted to the second area in a gel form or a semifluidic form.

In some embodiments, the concentration of the diffusion-restricted nuclease in the solution is at least about 0.0001 mg/mL, at least about 0.001 mg/mL, at least about 0.01 mg/mL, at least about 0.02 mg/mL, at least about 0.03 mg/mL, at least about 0.04 mg/mL, at least about 0.05 mg/mL, at least about 0.06 mg/mL, at least about 0.07 mg/mL, at least about 0.08 mg/mL, at least about 0.09 mg/mL, at least about 0.1 mg/mL, at least about 0.2 mg/mL, at least about 0.3 mg/mL, at least about 0.4 mg/mL, at least about 0.5 mg/mL, at least about 0.6 mg/mL, at least about 0.7 mg/mL, at least about 0.8 mg/mL, at least about 0.9 mg/mL, at least about 1 mg/mL, at least about 2 mg/mL, at least about 3 mg/mL, at least about 4 mg/mL, at least about 5 mg/mL, at least about 6 mg/mL, at least about 7 mg/mL, at least about 8 mg/mL, at least about 9 mg/mL, at least about 10 mg/mL, at least about 20 mg/mL, at least about 30 mg/mL, at least about 40 mg/mL, at least about 50 mg/mL, at least about 60 mg/mL, at least about 70 mg/mL, at least about 80 mg/mL, at least about 90 mg/mL, at least about 100 mg/mL, at least about 200 mg/mL, at least about 500 mg/maL, at least about 800 mg/mL, or at least about 1000 mg/mL.

In some embodiments, the concentration of the diffusion-restricted nuclease in the solution is at least about 0.001 units/mL, at least about 0.01 units/mL, at least about 0.02 units/mL, at least about 0.03 units/mL, at least about 0.04 units/mL, at least about 0.05 units/mL, at least about 0.06 units/mL, at least about 0.07 units/mL, at least about 0.08 units/mL, at least about 0.09 units/mL, at least about 0.1 units/mL, at least about 0.2 units/mL, at least about 0.3 units/mL, at least about 0.4 units/mL, at least about 0.5 units/mL, at least about 0.6 units/mL, at least about 0.7 units/mL, at least about 0.8 units/mL, at least about 0.9 units/mL, at least about 1 unit/mL, at least about 2 units/mL, at least about 3 units/mL, at least about 4 units/mL, at least about 5 units/mL, at least about 6 units/mL, at least about 7 units/mL, at least about 8 units/mL, at least about 9 units/mL, at least about 10 units/mL, at least about 20 units/mL, at least about 30 units/mL, at least about 40 units/mL, at least about 50 units/mL, at least about 60 units/mL, at least about 70 units/mL, at least about 80 units/mL, at least about 90 units/mL, or at least about 100 units/mL.

In some embodiments, the second area of the array can be contacted by the solution for at least about 5 minutes, at least about 10 minutes, at least about 15 minutes, at least about 20 minutes, at least about 25 minutes, at least about 30 minutes, at least about 40 minutes, at least about 50 minutes, or at least about 1 hour, or greater at a temperature of about 4° C., about 10° C., about 20° C., about 25° C., about 30° C., about 32° C., about 34° C., about 35° C., about 36° C., about 37° C., about 38° C., about 39° C., about 40° C., about 45° C., or about 50° C.

In some embodiments, the second area of the array can be contacted by the solution for about 5 minutes to about 1 hour, about 5 minutes to about 50 minutes, about 5 minutes to about 40 minutes, about 5 minutes to about 30 minutes, about 5 minutes to about 20 minutes, about 5 minutes to about 10 minutes, about 10 minutes to about 1 hour, about 10 minutes to about 50 minutes, about 10 minutes to about 40 minutes, about 10 minutes to about 30 minutes, about 10 minutes to about 20 minutes, about 20 minutes to about 1 hour, about 20 minutes to about 50 minutes, about 20 minutes to about 40 minutes, about 20 minutes to about 30 minutes, about 30 minutes to about 1 hour, about 30 minutes to about 50 minutes, about 30 minutes to about 40 minutes, about 40 minutes to about 1 hour, about 40 minutes to about 50 minutes, or about 50 minutes to about 1 hour, at a temperature of about 4° C. to about 35° C., about 4° C. to about 30° C., about 4° C. to about 25° C., about 4° C. to about 20° C., about 4° C. to about 15° C., about 4° C. to about 10° C., about 10° C. to about 35° C., about 10° C. to about 30° C., about 10° C. to about 25° C., about 10° C. to about 20° C., about 10° C. to about 15° C., about 15° C. to about 35° C., about 15° C. to about 30° C., about 15° C. to about 25° C., about 15° C. to about 20° C., about 20° C. to about 35° C., about 20° C. to about 30° C., about 20° C. to about 25° C., about 25° C. to about 35° C., about 25° C. to about 30° C., or about 30° C. to about 35° C.

### IV. Removing the Diffusion-Restricted Nuclease from the Second Area of the Array

In some embodiments, the solution comprising a diffusion-restricted nuclease is removed by pipetting. In some embodiments, the diffusion-restricted nuclease is removed by wicking (e.g., by an absorption paper). In some embodiments, the diffusion-restricted nuclease is removed by washing (e.g., using a wash buffer). In some embodiments, the wash buffer can be added to contact the second area of the array then removed by pipetting, wicking, or other methods known in the art. In some embodiments, a combination of removing methods can be used. In some embodiments, contacting and removing steps can be repeated (e.g., at least 2 times, 3 times, 4 times, or greater). In some embodiments, a drying step can be performed after washing (e.g., air dry).

In some embodiments, the wash buffer is added automatically (e.g., by a robot) or manually (e.g., by pipetting). In some embodiments, the wash buffer is added vertically above the second area of the array. In some embodiments, the wash buffer is added dropwise by a pipette. In some embodiments, the wash buffer is added to contact all or a portion of the second area of the array. In some embodiments, the wash buffer is added to all or a portion of a surface of the non-permeabilized biological sample that is not facing or contacting the array.

In some embodiments, the wash buffer comprises a nuclease inhibitor so as to stop the diffusion-restricted nuclease from functioning. In some embodiments, the washing buffer is 1X TE buffer, 1X TAE buffer, 1X TBE buffer, or PBS. In some embodiments, the wash buffer contains a buffer (e.g., Tris, MOPS, HEPES, MES, or any other buffer known in the art), chelating agents (e.g., ethylenediaminetetraacetic acid (EDTA)) and/or metal ions (e.g., Mg²⁺).

In some embodiments, the wash buffer can have a pH that is about 5.0, about 5.5, about 6.0, about 6.5, about 7.0, about 7.5, about 8.0, about 8.5, about 9.0, about 9.5, or about 10.0, or about 5.0 to 5.5, about 5.5 to 6.0, about 6.0 to 6.5, about 6.5 to 7.0, about 7.0 to 7.5, about 7.5 to 8.0, about 8.0 to 8.5, about 8.5 to 9.0, about 9.0 to 9.5, or about 9.5 to 10.0.

In some embodiments, the second area of the array is contacted by the wash buffer for about 5 seconds to about 1 hour, about 5 seconds to about 50 minutes, about 5 seconds to about 40 minutes, about 5 seconds to about 30 minutes, about 5 seconds to about 20 minutes, about 5 seconds to about 10 minutes, about 5 seconds to about 5 minutes, about 5 seconds to about 1 minute, about 5 seconds to about 30 seconds, about 5 seconds to about 10 seconds, about 10 seconds to about 1 hour, about 10 seconds to about 50 minutes, about 10 seconds to about 40 minutes, about 10 seconds to about 30 minutes, about 10 seconds to about 20 minutes, about 10 seconds to about 10 minutes, about 10 seconds to about 5 minutes, about 10 seconds to about 1 minute, about 10 seconds to about 30 seconds, about 30 seconds to about 1 hour, about 30 seconds to about 50 minutes, about 30 seconds to about 40 minutes, about 30 seconds to about 30 minutes, about 30 seconds to about 20 minutes, about 30 seconds to about 10 minutes, about 30 seconds to about 5 minutes, about 30 seconds to about 1 minute, about 1 minute to about 1 hour, about 1 minute to about 50 minutes, about 1 minute to about 40 minutes, about 1 minute to about 30 minutes, about 1 minute to about 20 minutes, about 1 minute to about 10 minutes, about 1 minute to about 5 minutes, about 5 minutes to about 1 hour, about 5 minutes to about 50 minutes, about 5 minutes to about 40 minutes, about 5 minutes to about 30 minutes, about 5 minutes to about 20 minutes, about 5 minutes to about 10 minutes, about 10 minutes to about 1 hour, about 10 minutes to about 50 minutes, about 10 minutes to about 40 minutes, about 10 minutes to about 30 minutes, about 10 minutes to about 20 minutes, about 20 minutes to about 1 hour, about 20 minutes to about 50 minutes, about 20 minutes to about 40 minutes, about 20 minutes to about 30 minutes, about 30 minutes to about 1 hour, about 30 minutes to about 50 minutes, about 30 minutes to about 40 minutes, about 40 minutes to about 1 hour, about 40 minutes to about 50 minutes, or about 50 minutes to about 1 hour at a temperature of about 4° C. to about 35° C., about 4° C. to about 30° C., about 4° C. to about 25° C., about 4° C. to about 20° C., about 4° C. to about 15° C., about 4° C. to about 10° C., about 10° C. to about 35° C. to about 10° C. to about 30° C., about 10° C. to about 25° C., about 10° C. to about 20° C., about 10° C. to about 15° C., about 15° C. to about 35° C., about 15° C. to about 30° C., about 15° C. to about 25° C., about 15° C. to about 20° C., about 20° C. to about 35° C., about 20° C. to about 30° C., about 20° to about 25° C., about 25° C. to about 35° C., about 25° C. to about 30° C., or about 30° C. to about 35° C.

In some embodiments, the second area of the array can be contacted by the wash buffer for at least about 1 second, at least about 5 seconds, at least about 10 seconds, at least about 15 seconds, at least about 20 seconds, at least about 30 seconds, at least about 45 seconds, at least about 1 minutes, at least about 5 minutes, at least about 10 minutes, at least about 15 minutes, at least about 20 minutes, at least about 25 minutes, at least about 30 minutes, at least about 40 minutes, at least about 50 minutes, at least about 1 hour, at a temperature of about 4° C., about 10° C., about 20° C., about 25° C., about 30° C., about 32° C., about 34° C., about 35° C., about 36° C., about 37° C., about 38° C., about 39° C., about 40° C., about 45° C. or about 50° C.

In some embodiments, the solution comprising the diffusion-restricted nuclease contains a gel precursor material (e.g., polyacrylamide) and the diffusion-restricted nuclease is removed by first adding a solution comprising a cross-linking agent (e.g., APS/TEMED) to polymerize or gel the gel precursor material, followed by separating the solution present as a gel from the second area of the array.

In some embodiments, the solution comprising the diffusion-restricted nuclease is present as a gel, and the solution can be removed by separating the gel from the second area of the array.

In some embodiments, the solution comprising the diffusion-restricted nuclease is removed by reducing the temperature of the solution to no more than about 0° C., no more than about −5° C., no more than about −10° C., no more than about −20° C., no more than about −50° C., no more than about −80° C. (e.g., lower than the melting point of the solution), followed by separating the solution in solid form from the second area of the array.

In some embodiments, the diffusion-restricted nuclease is linked to a magnetic bead (or a magnetic particle, or other magnetic substance thereof) and the diffusion-restricted nuclease can be removed by applying a magnetic field.

### V. Methods for Determining a Location of a Target Nucleic Acid

Provided herein are methods for determining a location of a target nucleic acid in a biological sample disposed onto an array (e.g., any of the arrays described herein), where the array has a first area covered by the biological sample (e.g., any of the first areas described herein) and a second area not covered by the biological sample (e.g., any of the second areas described herein); where the array comprises a plurality of capture probes (e.g., any of the capture probes described herein), where a capture probe of the plurality of capture probes comprises a spatial barcode (e.g., any of the spatial barcodes described herein) and a capture domain (e.g., any of the capture domains as described herein), where the methods include: (a) contacting the second area of the array with a solution comprising a diffusion-restricted nuclease (e.g., any of the diffusion-restricted nucleases described herein); (b) removing the diffusion-restricted nuclease from the second area of the array (e.g., using any of the methods for removing the diffusion-restricted nucleases described herein); (c) permeabilizing the biological sample, such that the capture domain binds to the target nucleic acid (e.g., using any of the methods for permeabilizing a biological sample described herein); and (d) determining (i) all or a portion of the sequence of the spatial barcode, or a complement thereof, and (ii) all or a portion of the sequence of the target nucleic acid, or a complement thereof, and using the sequences of (i) and (ii) to determine the location of the target nucleic acid in the biological sample.

Non-limiting examples of a target nucleic acid include DNA analytes such as genomic DNA, methylated DNA, specific methylated DNA sequences, fragmented DNA, mitochondrial DNA, in situ synthesized PCR products, and RNA/DNA hybrids.

Non-limiting examples of the target nucleic acid also include RNA analytes such as various types of coding and non-coding RNA. Examples of the different types of RNA analytes include messenger RNA (mRNA), ribosomal RNA (rRNA), transfer RNA (tRNA), microRNA (miRNA), and viral RNA. The RNA can be a transcript (e.g., present in a tissue section). The RNA can be small (e.g., less than 200 nucleic acid bases in length) or large (e.g., RNA greater than 200 nucleic acid bases in length). Small RNAs mainly include 5.8 S ribosomal RNA (rRNA), 5 S rRNA, transfer RNA (RNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16 s rRNA or 23 s rRNA).

In some embodiments, the target nucleic acid can include at least 1, at least 2, at least 3, at least 4, at least 5, at least 6, at least 7, at least 8, at least 9, or at least 10 disease-causing mutations (e.g., cancer-causing mutations). In some embodiments, the target nucleic acid includes polymorphism, gene amplification, or chromosomal translocation.

In some embodiments, the biological sample as described herein can be stained or imaged by techniques known in the art. In some embodiments, the staining includes optical labels as described herein, including, but not limited to, fluorescent, radioactive, chemiluminescent, calorimetric, or colorimetric detectable labels. In some embodiments, the staining includes a fluorescent antibody directed to a target analyte (e.g., cell surface or intracellular proteins) in the biological sample. In some embodiments, the staining includes an immunohistochemistry stain directed to a target analyte (e.g., cell surface or intracellular proteins) in the biological sample. In some embodiments, the staining includes a chemical stain such as hematoxylin and eosin (H&E) or periodic acid-schiff (PAS). In some embodiments, significant time (e.g., days, months, or years) can elapse between staining and/or imaging the biological sample and performing analysis.

### VI. Methods for Determining a Location of a Target Analyte

Provided herein are methods for determining a location of a target analyte in a biological sample that include: (a) contacting a plurality of analyte capture agents (e.g., any of the analyte capture agents described herein) to the biological sample, where an analyte capture agent of the plurality of analyte capture agents comprises an analyte binding moiety barcode (e.g., any of the analyte binding moiety barcodes described herein), an analyte capture sequence (e.g., any of the analyte capture sequences described herein), and an analyte binding moiety (e.g., any of the analyte binding moieties described herein) that binds specifically to the target analyte; (b) disposing the biological sample onto an array (e.g., any of the arrays described herein), where the array has a first area (e.g., any of the first areas described herein) covered by the biological sample and a second area (e.g., any of the second areas described herein) not covered by the biological sample, where the array comprises a plurality of capture probes, where a capture probe of the plurality comprises a spatial barcode (e.g., any of the spatial barcodes described herein) and a capture domain (e.g., any of the capture domains described herein) that binds specifically to the analyte capture sequence; (c) contacting the second area of the array with a solution comprising a diffusion-restricted nuclease (e.g., any of the diffusion-restricted nucleases described herein); (d) removing the diffusion-restricted nuclease from the second area of the array (e.g., using any of the methods for removing the diffusion-restricted nucleases described herein); and (e) determining (i) all or a portion of the sequence of the spatial barcode, or a complement thereof, and (ii) all or a portion of the sequence of the analyte binding moiety barcode, or a complement thereof, and using the sequences of (i) and (ii) to determine the location of the target analyte in the biological sample.

### VII. Methods for Decreasing the Background binding of a Target Nucleic Acid or Target Analyte

Provided herein are methods for decreasing background binding of a target nucleic acid on an array that include: (a) disposing a biological sample onto an array (e.g., any of the arrays described herein), wherein the array has a first area (e.g., any of the first areas described herein) covered by the biological sample and a second area (e.g., any of the second areas described herein) not covered by the biological sample, wherein the array comprises a plurality of capture probes (e.g., any of the capture probes described herein). wherein a capture probe of the plurality of capture probes comprises a spatial barcode (e.g., any of the spatial barcodes described herein) and a capture domain (e.g., any of the capture domains described herein); (b) contacting the second area of the array with a solution comprising a diffusion-restricted nuclease (e.g., any of the diffusion-restricted nucleases described herein), (c) removing the diffusion-restricted nuclease from the second area of the array (e.g., using any of the methods for removing the diffusion-restricted nucleases described herein); and (d) permeabilizing the biological sample (e.g., using any of the methods for permeabilizing a biological sample described herein), such that the capture domain binds to the target nucleic acid in the first area, thereby decreasing the background binding of a target nucleic acid on the array. Some embodiments further include: (e) (e) determining (i) all or a portion of the sequence of the spatial barcode, or a complement thereof, and (ii) all or a portion of the sequence of the target nucleic acid, or a complement thereof, and using the sequences of (i) and (ii) to determine the location of the target nucleic acid in the biological sample.

Provided herein are methods for decreasing background binding of a target analyte on an array that include: (a) contacting a plurality of analyte capture agents (e.g., any of the analyte capture agents described herein) to the biological sample, wherein an analyte capture agent of the plurality of analyte capture agents comprises an analyte binding moiety barcode (e.g., any of the analyte binding moiety barcodes described herein), an analyte capture sequence (e.g., any of the analyte capture sequences described herein), and an analyte binding moiety (e.g., any of the analyte binding moieties described herein) that binds specifically to the target analyte; (b) disposing the biological sample onto an array (e.g., any of the arrays described herein), wherein the array has a first area (e.g., any of the first areas described herein) covered by the biological sample and a second area (e.g., any of the second areas described herein) not covered by the biological sample, wherein the array comprises a plurality of capture probes, wherein a capture probe of the plurality comprises a spatial barcode (e.g., any of the spatial barcodes described herein) and a capture domain (e.g., any of the capture domains described herein) that binds specifically to the analyte capture sequence; (c) contacting the second area of the array with a solution comprising a diffusion-restricted nuclease (e.g., any of the diffusion-restricted nucleases described herein); (d) removing the diffusion-restricted nuclease from the second area of the array (e.g., using any of the methods for removing the diffusion-restricted nucleases described herein), and (e) permeabilizing the biological sample (e.g., using any of the methods for permeabilizing a biological sample described herein), such that the capture domain binds to the analyte capture sequence in the first area, thereby decreasing the background binding of a target analyte on the array. In some embodiments, the methods further include: (f) determining (i) all or a portion of the sequence of the spatial barcode, or a complement thereof, and (ii) all or a portion of the sequence of the analyte binding moiety barcode, or a complement thereof, and using the sequences of (i) and (ii) to determine the location of the target analyte in the biological sample. In some embodiments of these methods, step (a) is performed before step (b). In some embodiments of these methods, step (b) is performed before step (a).

Provided herein are methods for decreasing the background binding of a target analyte on a spatial array. For example, as seen in FIGS. 1 and 2, the tissue sections are surrounded by, in these figures, background fluorescence. One cause of the background fluorescence could be a result of target analytes passively diffusing from the tissues and binding to the capture domains of the capture probes that are around the tissue (e.g., a second area of the array). The methods provided herein provide for the digestion, complete or partial, of the nucleic acid capture probes that are not under the biological sample (e.g., the first area). It is contemplated that by completely or partially rendering the capture probe domains adjacent to the biological sample inoperable (e.g., via digestion with an endonuclease or exonuclease, or both), binding of target analytes will be minimized or eliminated, thereby decreasing background on the spatial array. As such, methods for decreasing background on a spatial array comprise a biological sample disposed on a spatial array, wherein the spatial array has a first area covered by the biological sample and a second area not covered by the biological sample, wherein the spatial array comprises a plurality of capture probes, wherein a capture probe of the plurality of capture probes comprises a spatial barcode and a capture domain. The second area of the array is covered with a solution comprising a diffusion-restricted nuclease (any of the nucleases as described herein restricted by any means described herein), which is allowed to be in contact with the second area for a period of time to completely or partially digest all or a portion of the capture probe domain, after which point the nuclease is removed for the spatial array, for example by washing. Following nuclease treatment of the second area of the spatial array, it is contemplated that minimal binding of target analytes from the biological sample will occur. The biological sample can be permeabilized thereby releasing the target analytes from the tissues, target analytes which can bind to the capture domains in the first are and not the second area of the spatial array. The bound analytes can complete the spatial transcriptomics workflow, for example by extension of the capture probe to create complementary sequences to the spatial barcode and (at least) all or a portion of the target analyte sequence, sequences which can be determined to identify the target analyte and its location in the biological sample.

### VIII. Kits

Also provided herein are kits that include: an array comprising a plurality of capture probes (e.g., any of the exemplary capture probes described herein), where a capture probe of the plurality of capture probes comprises a spatial barcode (e.g., any of the exemplary spatial barcodes described herein) and a capture domain (e.g., any of the exemplary capture domains described herein); and a diffusion-restricted nuclease (e.g., any of the exemplary diffusion-restricted nucleases described herein).

Also provided herein are kits that include: a plurality of analyte capture agents (e.g., any of the exemplary analyte capture agents described herein), where an analyte capture agent of the plurality of analyte capture agents comprises an analyte binding moiety barcode (e.g., any of the exemplary analyte binding moiety barcodes described herein), an analyte capture sequence (e.g., any of the exemplary analyte capture sequences described herein), and an analyte binding moiety (e.g., any of the exemplary analyte binding moieties described herein) that binds specifically to a target analyte; an array comprising a spatial barcode (e.g., any of the exemplary spatial barcodes described herein) and a capture domain (e.g., any of the exemplary capture domains described herein) that binds specifically to the analyte capture sequence; and a diffusion-restricted nuclease (e.g., any of the exemplary diffusion-restricted nucleases described herein).

In some embodiments of any of the kits described herein, the diffusion-restricted nuclease degrades single-stranded nucleic acids. In some embodiments of any of the kits described herein, the diffusion-restricted nuclease degrades double-stranded nucleic acids. In some embodiments of any of the kits described herein, the diffusion-restricted nuclease is covalently linked to a bead (e.g., any of the exemplary beads described herein). In some embodiments of any of the kits described herein, the diffusion-restricted nuclease is covalently linked to a particle (e.g., any of the exemplary particles described herein). In some embodiments of any of the kits described herein, the diffusion-restricted nuclease is covalently linked to a polymer (e.g., any of the exemplary polymers described herein, such as a polyethylene glycol). In some embodiments of any of the kits described herein, the diffusion-restricted nuclease is an endonuclease. In some embodiments of any of the kits described herein, the diffusion-restricted nuclease is an exonuclease (e.g., a 3′ to 5′ exonuclease). In some embodiments of any of the kits described herein, the exonuclease is a DNAse. In some embodiments of any of the kits described herein, the array comprises a slide or a bead array.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method comprising:
(a) contacting a biological sample with a probe pair, wherein a first probe and a second probe of the probe pair comprise sequences that are substantially complementary to sequences in a target nucleic acid in the biological sample;
(b) hybridizing the first probe and the second probe to the target nucleic acid;
(c) generating a ligation product by ligating (i) the first probe and the second probe, or (ii) an extended first probe and the second probe;
(d) contacting the biological sample with a substrate comprising a plurality of capture probes affixed thereto, wherein each capture probe of the plurality of capture probes comprises:
(i) a spatial barcode that identifies a location of a given capture probe on the substrate; and
(ii) a capture domain, wherein the substrate comprises a first area in contact with the biological sample and a second area not in contact with the biological sample;
(e) contacting the second area of the substrate with a diffusion-restricted nuclease, thereby digesting one or more capture probes in the second area of the substrate;
(f) removing the diffusion-restricted nuclease from the second area of the substrate; and
(g) permeabilizing the biological sample such that a capture domain of a capture probe in the first area of the substrate binds to the ligation product.

2. The method of claim 1, wherein the first probe and the second probe are substantially complementary to adjacent sequences in the target nucleic acid.

3. The method of claim 1, wherein the first probe and the second probe hybridize to sequences that are not adjacent to each other in the target nucleic acid.

4. The method of claim 3, further comprising extending the first probe with a polymerase, thereby (i) filling in a gap between the first probe and the second probe hybridized to the target nucleic acid and (ii) generating the extended first probe.

5. The method of claim 1, wherein generating the ligation product comprises ligating the first probe and the second probe using enzymatic ligation.

6. The method of claim 5, wherein the enzymatic ligation utilizes a ligase.

7. The method of claim 1, wherein the second probe comprises a sequence that is complementary to the capture domain of the capture probe in the first area of the substrate.

8. The method of claim 1, wherein the first probe and/or the second probe is a DNA probe.

9. The method of claim 1, wherein the diffusion-restricted nuclease is covalently linked to a bead, a particle, or a polymer.

10. The method of claim 9, wherein the polymer is a polyethylene glycol.

11. The method of claim 1, wherein the removing the diffusion-restricted nuclease from the second area of the substrate in (f) comprises washing.

12. The method of claim 1, further comprising determining and/or obtaining (i) a sequence of the spatial barcode of the capture probe in the first area of the substrate or a complement thereof, and (ii) all or a portion of a sequence of the ligation product or a complement thereof.

13. The method of claim 12, wherein the determining comprises extending the capture probe in the first area of the substrate using the ligation product as a template.

14. The method of claim 12, further comprising identifying the location of the target nucleic acid in the biological sample using (i) the sequence of the spatial barcode of the capture probe in the first area of the substrate or a complement thereof, and (ii) all or a portion of the sequence of the ligation product or a complement thereof.

15. The method of claim 1, wherein the biological sample is a tissue section.

16. The method of claim 1, wherein the target nucleic acid comprises RNA or DNA.

17. The method of claim 1, wherein the target nucleic acid is conjugated to an antibody or an antibody fragment.

18. The method of claim 1, wherein the permeabilizing comprises contacting the biological sample with a permeabilization agent.

19. The method of claim 18, wherein the permeabilization agent comprises proteinase K or pepsin.

20. The method of claim 1, further comprising staining and/or imaging the biological sample.
